(19) United States
(12) Patent Application Publication
(10) Pub. No.: US 2010/0120888 A1
(43) Pub. Date: May 13, 2010

(54) ATORVASTATIN STRONTIUM SALT AND PHARMACEUTICAL COMPOSITION COMPRISING SAME

(75) Inventors: Eun Sook Kim, Seoul (KR); Sun Young Jang, Suwon-si (KR); Bo Sung Kwon, Yongin-si (KR); Sangmin Yun, Seongnam-shi (KR); Kwee Hyun Suh, Suwon-si (KR); Gwan Sun Lee, Seoul (KR)

Correspondence Address: SUGHRUE MION, PLLC, 2100 PENNSYLVANIA AVENUE, N.W., SUITE 800, WASHINGTON, DC, 20037, US

(73) Assignee: HANMI PHARM. CO., LTD., Kyungki-do (KR)

(21) Appl. No.: 12/524,807

(22) PCT Filed: Jan. 21, 2008
(86) PCT No.: PCT/KR2008/000357

§ 371 Date: Jul. 28, 2009

(30) Foreign Application Priority Data
Jan. 29, 2007 (KR) 10-2007-0008896

(51) Int. Cl. A61K 31/40; C07D 207/323; A61P 3/06; A61P 3/00

(52) U.S. Cl. 514/423; 548/537

(57) ABSTRACT

This invention provides atorvastatin strontium salt or its hydrates or polymorphs having improved water solubility, which is useful for the prevention or treatment of hyperlipidemia and hypercholesterolemia, and a pharmaceutical composition comprising same.

### FIELD OF THE INVENTION

The present invention relates to atorvastatin strontium salt or its hydrates or polymorphs having improved solubility and a pharmaceutical composition comprising same.

### DESCRIPTION OF THE PRIOR ART

Atorvastatin, [R—(R*,R*)]-2-(4-fluorophenyl)-β,δ-dihydroxy-5-(1-methylethyl)-3-phenyl-4-[(phenylamino)carbonyl]-1H-pyrrole-1-heptanoic acid having the structure of formula (II) is known as an HMG-CoA reductase inhibitor which is effective in reducing cholesterol in blood (see U.S. Pat. No. 5,273,995).

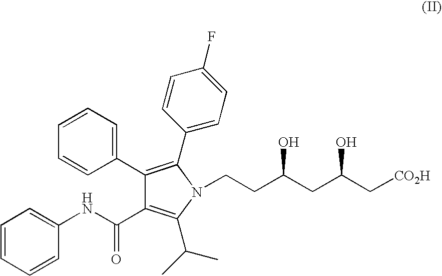

The free acid form of atorvastatin has low stability and tends to convert to atorvastatin lactone of formula (III). Accordingly, in the preparation of pharmaceutical composition of atorvastatin, various salts of atorvastatin have been used instead of the free acid form. U.S. Pat. No. 5,273,995 discloses various atorvastatin salts, e.g., metal salts such as sodium, potassium, lithium, calcium, magnesium, zinc, aluminum and iron (ferric or ferrous) salts and organic salts such as 1-deoxy-2-(methylamino)-D-glucitol, N-methylglucamine, choline and arginine salts, among which atorvastatin calcium salt of formula (IV) is most preferred.

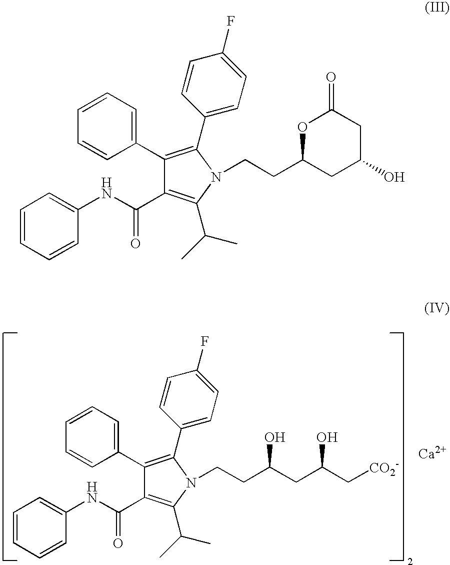

Other atorvastatin salts have also been suggested: t-butylamine and dicyclohexylamine salts (PCT International Publication No. WO 2000/017150); ammonium salt (WO 2001/036384); lysine, arginine and ornithine salts (WO 2003/082816) and bismuth salt (WO 2005/014541). However, these salts are inferior to the calcium salt of atorvastatin in terms of various pharmaceutical characteristics including hygroscopicity and stability. The atorvastatin calcium salt of formula (IV) including its hydrate or solvate can exist in various crystalline forms designated Forms I, II and IV (U.S. Pat. No. 5,969,156), Form III (U.S. Pat. No. 6,121,461), and Forms V and XIX (U.S. Pat. No. 6,605,729). Other crystalline forms of atorvastatin calcium salt have been disclosed in PCT International Publication Nos. WO 2003/070702, WO 2004/043918 and WO 2006/048894. Also, various forms of amorphous atorvastatin calcium salt have been disclosed in PCT International Publication Nos. WO 1997/003960, WO 2000/071116, WO 2001/028999, WO 2001/042209, WO 2003/068739, WO 2005/073187 and WO 2006/021969. However, the amorphous forms of atorvastatin are difficult to produce in lager-scales and they are inferior to the crystalline forms in terms of physicochemical properties such as hygroscopicity, stability, in vivo uptake rate and bioavailability (see Oishi and Yakuri, Chiryo, 1998, 26(8), 1241-1252).

Among the known atorvastatin calcium salts, crystalline or amorphous, crystalline Form I of atorvastatin calcium salt disclosed in U.S. Pat. No. 5,969,156 has been considered to be most preferred for commercial applications, which is marketed under the trade name Lipitor® (Pfizer) as a therapeutic agent for hyperlipidemia.

When a pharmaceutical composition is formulated, the physicochemical properties of the solid form of an active ingredient affect the flowability during solid milling, the compressibility during tablet formulation, or the release rate in an aqueous media. Particularly, in case of a pharmaceutical composition for oral administration, the release rate of the active ingredient into the gastrointestinal fluid is one of the important factors that determine the therapeutic effects. Thus, the solid form of the active ingredient of a pharmaceutical composition is required to have physicochemical properties that can improve its solubility, bioavailability, compressibility and stability, but such physicochemical properties of the known atorvastatin calcium salts are not entirely satisfactory

Therefore, there has been a need to develop a novel salt of atorvastatin having improved physicochemical properties and the present inventors have found that atorvastatin strontium salt or a hydrate thereof has enhanced physicochemical properties, particularly in terms of water solubility.

### SUMMARY OF THE INVENTION

It is a primary object of the present invention to provide atorvastatin strontium salt or its hydrates or polymorphs.

In accordance with one aspect of the present invention, there is provided atorvastatin strontium salt of formula (I) or its hydrates or polymorphs:

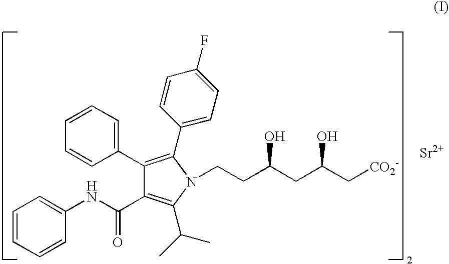

In accordance with another aspect of the present invention, there is provided a pharmaceutical composition comprising atorvastatin strontium salt of formula (I) or its hydrate or polymorph as an active ingredient and a pharmaceutically acceptable carrier for preventing or treating hyperlipidemia and hypercholesterolemia.

### BRIEF DESCRIPTION OF THE DRAWINGS

The above and other objects and features of the present invention will become apparent from the following description of the invention taken in conjunction with the accompanying drawings, which respectively show:

FIG. 1: an X-ray powder diffraction (XRPD) spectrum of crystalline Form I of atorvastatin strontium salt obtained according to the present invention;

FIG. 2: an XRPD spectrum of crystalline Form II of atorvastatin strontium salt obtained according to the present invention;

FIG. 3: an XRPD spectrum of crystalline Form III of atorvastatin strontium salt obtained according to the present invention;

FIG. 4: an XRPD spectrum of crystalline Form IV of atorvastatin strontium salt obtained according to the present invention; and

FIG. 5: an XRPD spectrum of the amorphous atorvastatin strontium salt obtained according to the present invention.

### DETAILED DESCRIPTION OF THE INVENTION

The inventive atorvastatin strontium salt of formula (I) or a hydrate or polymorph thereof is pure and thermally stable, and it is more soluble in an aqueous medium than any of the known atorvastatin salts.

The atorvastatin strontium salt according to the present invention has two atorvastatin molecules coordinated to strontium ion (II), to which at least one H₂O molecule may be coordinated. Such atorvastatin strontium salt or a hydrate thereof can be produced in an amorphous form or various crystalline forms.

In accordance with a preferred embodiment of the present invention, there are provided crystalline atorvastatin strontium salts or hydrates designated Forms I to IV, among these, Form I of atorvastatin strontium pentahydrate represented by formula (Ia) is preferred:

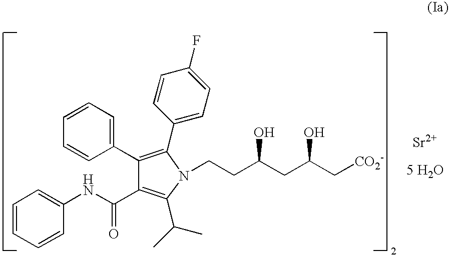

The crystallinity of the atorvastatin strontium salt of the present invention may be confirmed by X-ray powder diffraction (XRPD) analysis using CuKₐ radiation.

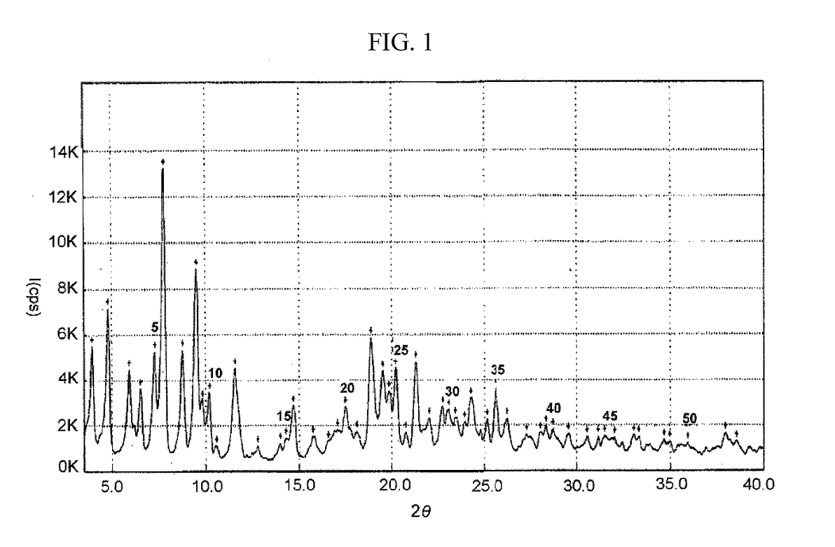

Specifically, the crystalline atorvastatin strontium pentahydrate of formula (Ia), which is designated Form I according to a preferred embodiment of the present invention, has a characteristic crystalline structure whose XRPD spectrum shows major peaks having I/Iₒ values of at least 10% (I is the intensity of each peak; Iₒ is the intensity of the highest peak) at diffraction angle (2θ±0.2) of 4.0, 4.8, 5.9, 6.5, 7.3, 7.8, 8.8, 9.5, 9.8, 10.2, 11.6, 14.7, 17.5, 18.9, 19.5, 19.8, 20.2, 21.3, 22.7, 23.1, 24.3, 25.6 and 26.3 (FIG. 1).

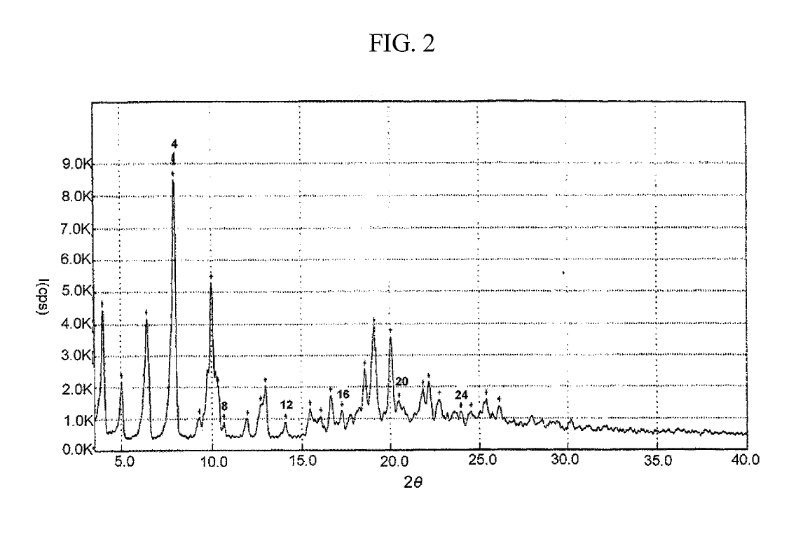

The crystalline atorvastatin strontium salt or its hydrate designated Form II according to another preferred embodiment of the present invention has a characteristic crystalline structure whose XRPD spectrum shows major peaks having I/Iₒ values of at least 10% at diffraction angle (2θ±0.2) of 4.0, 5.0, 6.4, 8.0, 10.0, 10.3, 12.7, 13.0, 16.6, 18.6, 19.1, 20.0, 21.8 and 22.2 (FIG. 2).

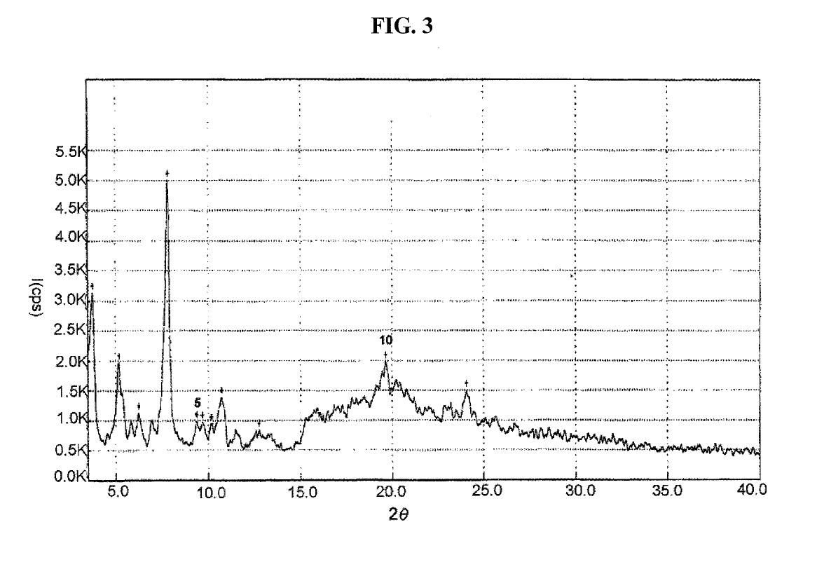

The crystalline atorvastatin strontium salt or its hydrate, which is designated Form III according to still another preferred embodiment of the present invention, has a characteristic crystalline structure whose XRPD spectrum shows major peaks having I/Iₒ values of at least 10% at diffraction angle (2θ±0.2) of 3.8, 5.2, 6.2, 7.9, 10.7, 19.7 and 24.0 (FIG. 3).

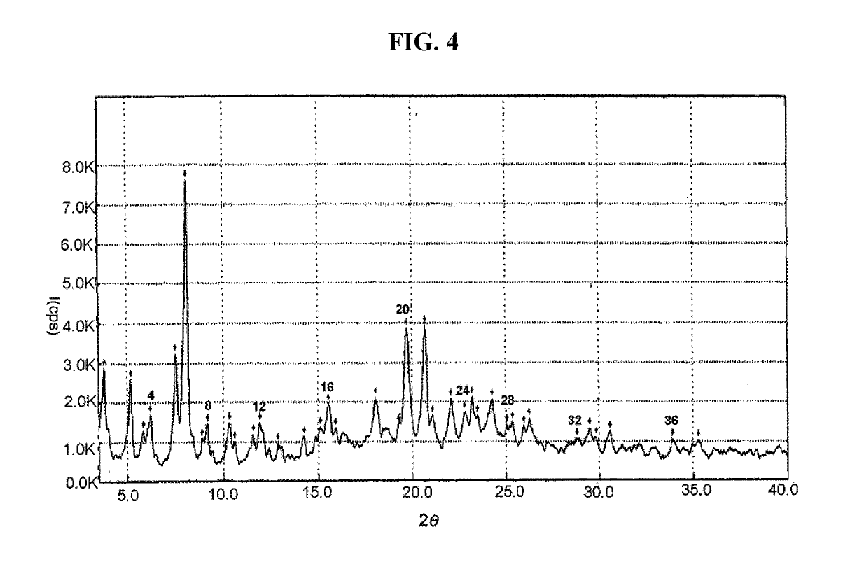

The crystalline atorvastatin strontium salt or its hydrate, which is designated Form IV according to still another preferred embodiment of the present invention, has a characteristic crystalline structure whose XRPD spectrum shows major peaks having I/Iₒ values of at least 10% at diffraction angle (2θ±0.2) of 3.8, 5.2, 5.8, 6.2, 7.6, 8.1, 9.2, 10.3, 11.9, 15.5, 18.1, 19.8, 20.7, 21.1, 22.1, 23.2, 24.3 and 26.3 (FIG. 4).

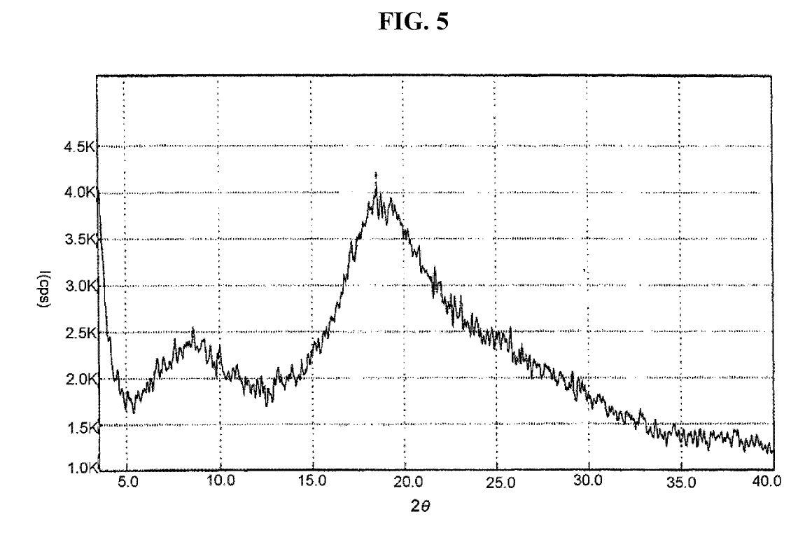

In accordance with yet another preferred embodiment of the present invention, amorphous atorvastatin strontium salt or its hydrate is also provided, and its XRD spectrum shows no distinctively characteristic peak (FIG. 5).

The inventive atorvastatin strontium salt of formula (I) or a hydrate or polymorph thereof may be obtained in a pure form and it satisfies the pharmaceutically required stability since it can maintain its initial moisture content when exposed to a stressed condition (60° C. and 75% relative humidity) for 4 weeks or more.

Further, the inventive atorvastatin strontium salt of formula (I) or a hydrate or polymorph thereof is pharmaceutically more effective because of its high water solubility over other salts of atorvastatin. For example, it has a water solubility at least 2 times higher than that of atorvastatin calcium trihydrate.

In accordance with the present invention, atorvastatin strontium salt of formula (I) or a hydrate or polymorph thereof may be prepared by (i) bringing atorvastatin of formula (II) or atorvastatin lactone of formula (III) to react with strontium hydroxide; or (ii) adding a reactive strontium salt to atorvastatin sodium or potassium salt to induce salt exchange; or (iii) converting one polymorphic form of the atorvastatin strontium salt or a hydrate thereof previously obtained to other desired polymorphic form.

Specifically, the atorvastatin strontium pentahydrate of formula (Ia) designated crystalline Form I may be prepared by adding a reactive strontium salt to a solution of atorvastatin sodium or potassium dissolved in a mixture of an organic solvent and water, stirring the resulting mixture at a temperature ranging from 0° C. to the boiling point of the solvent used for 30 minutes to 24 hours, filtering and drying the resulting precipitates by a conventional method.

The reactive strontium salt may be selected from strontium chloride, strontium bromide, strontium sulfate, strontium nitrate, strontium perchlorate, strontium acetate, strontium carbonate, strontium oxalate and a mixture thereof, preferably strontium chloride and strontium acetate.

The organic solvent may be selected from acetone, methyl ethyl ketone, methyl isobutyl ketone, acetonitrile, methanol, ethanol, isopropanol and a mixture thereof, preferably, acetonitrile, acetone and methanol.

The inventive atorvastatin strontium salt or a hydrate or polymorph thereof has good purity and thermal stability as well as improved water solubility, so that it can be pharmaceutically used for the prevention or treatment of HMG-CoA reductase-related diseases including hyperlipidemia and hypercholesterolemia.

Accordingly, the present invention provides a pharmaceutical composition comprising the inventive atorvastatin strontium salt of formula (I) or a hydrate or polymorph thereof as an active ingredient and a pharmaceutically acceptable carrier.

The pharmaceutical composition according to the present invention may be administered via various routes including oral, rectal and injectable application, preferably the oral route.

For oral administration, the pharmaceutical composition of the present invention may be in the form of tablets, capsules, pills, and the like, and may be formulated with pharmaceutically acceptable carriers, diluents or excipients. Examples of suitable carriers, diluents and excipients are excipients such as starches, sugar and mannitol; filling agents or increasing agents such as calcium phosphate and silica derivatives; binding agents such as cellulose derivatives of carboxymethylcellulose or hydroxypropylcellulose, gelatin, arginic acid salt, and polyvinylpyrrolidone; lubricating agents such as talc, magnesium or calcium stearate, hydrogenated castor oil and solid polyethylene glycol; disintegrants such as povidone, croscarmellose sodium, and crospovidone; and surfactants such as polysorbate, cetyl alcohol and glycerol monostearate. Further, various pharmaceutical composition comprising a specific amount of active ingredient, together with or without additives such as said excipients, diluents or additives, may be prepared in accordance with any of the conventional procedures (see Remington's Pharmaceutical Science, Mack Publishing Company, Easton, Pa., 19^{th} Edition, 1995).

In a preferred embodiment, the pharmaceutical composition for oral administration of the present invention may contain atorvastatin strontium salt of formula (I) or a hydrate or polymorph thereof in an amount ranging from 0.1 to 95% by weight, preferably 1 to 70% by weight based on the total weight of the composition.

A typical daily dose of atorvastatin strontium salt of formula (I) or a hydrate or polymorph thereof for a mammalian including human may range from about 0.5 to 500 mg/kg body weight, preferably 5 to 150 mg/kg body weight, and can be administered in a single dose or in divided doses.

The present invention will be described in further detail with reference to Examples. However, it should be understood that the present invention is not restricted by the specific Examples.

### Example 1

### Preparation of Crystalline Form I of Atorvastatin Strontium Salt

50.0 g of atorvastatin lactone was suspended in a mixture of 150 a of t-butyl methyl ether and 100 ml of acetone, and 3.7 g of strontium hydroxide dissolved in 200 ml of water was slowly added thereto over 30 minutes, and stirred at room temperature for 3 hours. After removing the organic layer, 150 ml of t-butyl methyl ether was added to the aqueous layer, followed by stirring at room temperature for 10 minutes. The organic layer was again removed, and 200 ml of acetone was added to the aqueous layer. The mixture was warmed to 50° C., to which 10.2 g of strontium acetate dissolved in 250 ml of water was slowly added over 2 hours, stirred at 50° C. for 8 hours, and the resulting solution was cooled to room temperature. The precipitate formed was filtered, washed with a mixture of 60 ml of acetone and 90 ml of water and dried in air, to obtain 50.7 g of the title compound (yield: 85%) as a white crystalline powder.

Moisture content (Karl-Fisher titrator): about 6.9%

Based on the result of such moisture content analysis, the crystalline powder obtained above was confirmed to be the pentahydrate form of formula (Ia), and its XRPD result showed that it is a crystal having distinctively characteristic main peaks (those having I/Iₒ of at least 10%), as shown in Table 1. Accordingly, the crystalline powder obtained above is designated Form I.

TABLE 1

| 2θ (±2) | d | I/Iₒ (%) |
|---|---|---|
| 4.0 | 22.3 | 32.7 |
| 4.8 | 18.3 | 49.4 |
| 5.9 | 14.9 | 29.1 |
| 6.5 | 13.5 | 21.7 |
| 7.3 | 12.1 | 35.0 |
| 7.8 | 11.3 | 100.0 |
| 8.8 | 10.1 | 35.4 |
| 9.5 | 9.3 | 64.7 |
| 9.8 | 9.0 | 18.2 |
| 10.2 | 8.7 | 21.6 |
| 11.6 | 7.6 | 31.6 |
| 14.7 | 6.0 | 18.8 |
| 17.5 | 5.1 | 14.1 |
| 18.9 | 4.7 | 37.9 |
| 19.5 | 4.5 | 25.9 |
| 19.8 | 4.5 | 19.0 |
| 20.2 | 4.4 | 27.5 |
| 21.3 | 4.2 | 30.2 |
| 22.7 | 3.9 | 12.2 |
| 23.1 | 3.9 | 10.7 |
| 24.3 | 3.7 | 14.5 |
| 25.6 | 3.5 | 19.6 |
| 26.3 | 3.4 | 10.3 |

2θ: diffraction angle, d: distance within each crystal face, I/I₀ (%): relative intensity of peak

### Example 2

### Preparation of Crystalline Form II of Atorvastatin Strontium Salt

10.0 g of crystalline Form I of atorvastatin strontium salt obtained in Example 1 was dried under a reduced pressure until its moisture content became 2% or less, to obtain 9.3 g of the title compound (yield: 100%) as a white crystalline powder.

Moisture content (Karl-Fisher titrator): about 1.5%

The XRPD result of the crystalline powder obtained above showed that the crystalline powder is a crystal having distinctively characteristic main peaks (those having I/Iₒ of at least 10%), as shown in Table 2. Accordingly, the crystalline powder is designated Form II.

TABLE 2

| 2θ (±2) | d | I/Iₒ (%) |
|---|---|---|
| 4.0 | 22.0 | 45.6 |
| 5.0 | 17.5 | 21.6 |
| 6.4 | 13.7 | 46.5 |
| 8.0 | 11.1 | 100.0 |
| 10.0 | 8.8 | 60.0 |
| 10.3 | 8.6 | 19.8 |
| 12.7 | 7.0 | 12.8 |
| 13.0 | 6.8 | 20.4 |
| 16.6 | 5.3 | 12.9 |
| 18.6 | 4.8 | 20.3 |
| 19.1 | 4.6 | 36.6 |
| 20.0 | 4.4 | 32.0 |
| 21.8 | 4.1 | 12.1 |
| 22.2 | 4.0 | 15.1 |

2θ: diffraction angle, d: distance within each crystal face, I/I₀ (%): relative intensity of peak

### Example 3

### Preparation of Crystalline Form III of Atorvastatin Strontium Salt

50.0 g of atorvastatin lactone was suspended in a mixture of 200 ml of t-butyl methyl ether and 200 ml of methanol, and 3.7 g of strontium hydroxide dissolved in 200 ml of water was slowly added thereto over 30 minutes, and stirred at room temperature for 3 hours. After removing the organic layer, 150 ml of t-butyl methyl ether was added to the aqueous layer, followed by stirring at room temperature for 10 minutes. The organic layer was again removed, and 50 ml of methanol, 150 ml of t-butyl methyl ether and 650 ml of distilled water were successively added to the aqueous layer. The mixture was warmed to 50° C., to which 10.2 g of strontium acetate dissolved in 250 ml of water was slowly added over 2 hours, stirred at 50° C. for 17 hours, and the resulting solution was cooled to room temperature. The precipitate formed was filtered, washed with a mixture of 100 ml of methanol and 50 ml of water and dried in air, to obtain 43 g of the title compound (yield: 77%) as a white crystalline powder.

Moisture content (Karl-Fisher titrator): about 5.5%

The XRPD result of the crystalline powder obtained above showed that the crystalline powder is a crystal having distinctively characteristic main peaks (those having I/Iₒ of at least 10%), as shown in Table 3. Accordingly, the crystalline powder is designated Form III.

TABLE 3

| 2θ (±2) | d | I/Iₒ (%) |
|---|---|---|
| 3.8 | 23.2 | 39.0 |
| 5.2 | 17.0 | 30.7 |
| 6.2 | 14.2 | 10.4 |
| 7.9 | 11.2 | 100.0 |
| 10.7 | 8.3 | 18.4 |
| 19.7 | 4.5 | 16.3 |
| 24.0 | 3.7 | 12.2 |

2θ: diffraction angle, d: distance within each crystal face, I/I₀ (%): relative intensity of peak

### Example 4

### Preparation of Crystalline Form IV of Atorvastatin Strontium Salt

4.5 g of crystalline Form III of atorvastatin strontium salt obtained in Example 3 was suspended in a mixture of 72 and of acetonitrile, 18 and of distilled water and 9 ml of t-butyl methyl ether, and the suspension was stirred at 55 to 60° C. for about 17 hours and cooled to room temperature. The precipitate formed was filtered and resuspended in a mixture of 30 in of acetonitrile and 30 ml of distilled water, and the suspension was stirred at 70° C. for 24 hours and cooled to room temperature. The precipitate formed was filtered and dried in air, to obtain 2.5 g of the title compound as a white crystalline powder.

Moisture content (Karl-Fisher titrator): about 4.2%

The XRPD result of the crystalline powder obtained above showed that the crystalline powder is a crystal having distinctively characteristic main peaks (those having I/Iₒ of at least 10%), as shown in Table 4. Accordingly, the crystalline powder is designated Form IV.

TABLE 4

| 2θ (±2) | d | I/Iₒ (%) |
|---|---|---|
| 3.8 | 23.1 | 24.2 |
| 5.2 | 17.0 | 27.9 |
| 5.8 | 15.1 | 10.4 |
| 6.2 | 14.2 | 16.6 |
| 7.6 | 11.7 | 38.5 |
| 8.1 | 10.9 | 100.0 |
| 9.2 | 9.6 | 13.5 |
| 10.3 | 8.6 | 14.2 |
| 11.9 | 7.4 | 13.1 |
| 15.5 | 5.7 | 16.4 |
| 18.1 | 4.9 | 16.5 |
| 19.8 | 4.5 | 40.2 |
| 20.7 | 4.3 | 42.1 |
| 21.1 | 4.2 | 10.6 |
| 22.1 | 4.0 | 16.0 |
| 23.2 | 3.8 | 14.8 |
| 24.3 | 3.7 | 14.3 |
| 26.3 | 3.4 | 10.1 |

2θ: diffraction angle, d: distance within each crystal face, I/I₀ (%): relative intensity of peak

### Example 5

### Preparation of Amorphous Form of Atorvastatin Strontium Salt

50.0 g of crystalline Form III of atorvastatin strontium salt obtained in Example 3 was dissolved in a mixture of 100 ml of acetone and 50 ml of methanol, while warming to 50° C. The resulting solution was filtered. To the filtrate, a mixture of 1,000 ml of t-butyl methyl ether and 500 ml of isopropyl ether was added in one portion. The mixture was stirred for 1 hour. The precipitate formed was filtered and dried in air, to obtain 45 g of the title compound (yield: 77%) as a white powder.

Moisture content (Karl-Fisher titrator): about 3.1%

The result of XRD analysis for the atorvastatin strontium salt obtained showed an amorphous form having no distinctively characteristic peak as shown in FIG. 5.

### Example 6

### Preparation of Crystalline Form I of Atorvastatin Strontium Salt

10.0 g of crystalline Form III of atorvastatin strontium salt obtained in Example 3 was suspended in a mixture of 100 ml of methanol and 100 ml of water, and the suspension was stirred at about 50° C. for about 17 hours and cooled to room temperature. The precipitate formed was filtered, washed with a mixture of 10 ml of methanol and 10 ml of water and dried in air, to obtain 9.2 g of the title compound (yield: 77%) as a white crystalline powder.

Moisture content (Karl-Fisher titrator): about 7.2%

The XRPD result of the compound obtained was the same as that of Example 1.

### Examples 7 to 11

### Preparation of Crystalline Form I of Atorvastatin Strontium Salt

The procedure of Example 6 was repeated except that 10.0 g of crystalline Form III of atorvastatin strontium salt obtained in Example 3 was suspended in water and an organic solvent as shown in Table 5, to obtain each of the title compounds.

TABLE 5

| | Organic solvent | Amount of | | Moisture |
|---|---|---|---|---|
| Ex. | (Amount, ml) | Water (ml) | Yield (%) | content (%) |
| 7 | Acetone (100) | 100 | 87 | 7.0 |
| 8 | Acetonitrile (100) | 100 | 74 | 6.9 |
| 9 | Acetone (50) | 50 | 88 | 6.8 |
| 10 | Isopropanol (50) | 50 | 90 | 7.1 |
| 11 | Methyl ethyl ketone (50) | 50 | 57 | 7.1 |

The XRPD results of the compounds obtained were the same as that of Example 1.

### Example 12

### Preparation of Crystalline Form I of Atorvastatin Strontium Salt

2.0 g of crystalline Form II of atorvastatin strontium salt obtained in Example 2 was placed in a chamber kept at a relative humidity of 60% for a period of one day or more, to obtain 2.1 g of the title compound as a white crystalline powder.

Moisture content (Karl-Fisher titrator): about 7.0%

The XRPD result of the compound obtained was the same as that of Example 1.

### Experimental Example 1

### Water-Solubility Test

The atorvastatin strontium salt or a hydrate or polymorph thereof prepared according to the present invention and the known atorvastatin calcium salt trihydrate were each dissolved in deionized water or in phosphoric acid buffer solution (pH 6.8) to saturation, and the remaining solid was removed by filtering. Each of the saturated solutions obtained after filtering was analyzed by HPLC according to the conditions measuring the amount of atorvastatin, to determine the amount of atorvastatin dissolved. The results are shown in Table 6.

TABLE 6

Solubility (mg/ml, 25° C.)

| | | Buffer solution |
|---|---|---|
| Salt | Deionized water | (pH 6.8) |
| Atorvastatin calcium | 0.14 | 0.26 |
| trihydrate* | | |
| Atorvastatin strontium | 0.38 | 0.45 |
| (Crystalline Form I) | | |
| Atorvastatin strontium | 0.38 | 0.50 |
| (Crystalline Form II) | | |
| Atorvastatin strontium | 0.25 | 0.28 |
| (Crystalline Form III) | | |
| Atorvastatin strontium | 0.46 | 0.33 |
| (Amorphous form) | | |

<Condition for HPLC analysis>
Detector: UV spectrometer at 248 nm
Column: Synerge Fusion RP80 (4.6 mm × 250 mm, 4 μm)
Column temperature: 40° C.
Elution condition: 0.1% phosphoric acid/acetonitrile = 55/45 (v/v)
Flow rate: about 1.5 ml/minute
*Atorvastatin calcium trihydrate is the salt designated as Form I in U.S. Pat. No. 5,969,156, which was prepared according to the procedure disclosed in such patent.

As shown in Table 6, the solubility of the inventive atorvastatin strontium salt or its hydrate or polymorph is at least 2 times higher than that of the known atorvastatin calcium trihydrate, which suggests that the inventive strontium salt or its hydrate or polymorph is more suitable for the release of atorvastatin.

While the invention has been described with respect to the specific embodiments, it should be recognized that various modifications and changes may be made by those skilled in the art to the invention which also fall within the scope of the invention as defined as the appended claims.

What is claimed is:

1. The atorvastatin strontium salt of formula (I) or its hydrates or polymorphs:

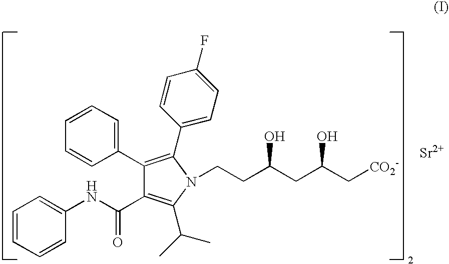

2. The atorvastatin strontium salt or its hydrates or polymorphs according to claim 1, which is a crystalline form.

3. The atorvastatin strontium salt or its hydrates or polymorphs according to claim 2, which is represented by formula (Ia):

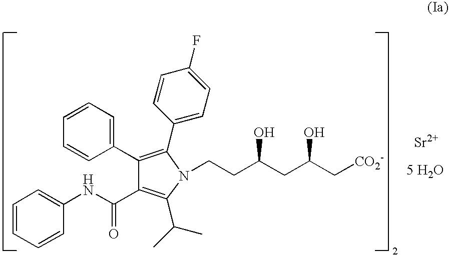

4. The atorvastatin strontium salt or its hydrates or polymorphs according to claim 3, whose X-ray powder diffraction spectrum shows major peaks having I/Iₒ values of at least 10% at 2θ±0.2 of 4.0, 4.8, 5.9, 6.5, 7.3, 7.8, 8.8, 9.5, 9.8, 10.2, 11.6, 14.7, 17.5, 18.9, 19.5, 19.8, 20.2, 21.3, 22.7, 23.1, 24.3, 25.6 and 26.3.

5. The atorvastatin strontium salt or its hydrates or polymorphs according to claim 2, whose X-ray powder diffraction spectrum shows major peaks having I/Iₒ values of at least 10% at 2θ±0.2 of 4.0, 5.0, 6.4, 8.0, 10.0, 10.3, 12.7, 13.0, 16.6, 18.6, 19.1, 20.0, 21.8 and 22.2.

6. The atorvastatin strontium salt or its hydrates or polymorphs according to claim 2, whose the X-ray powder diffraction spectrum shows major peaks having I/Iₒ values of at least 10% at 2θ±0.2 of 3.8, 5.2, 6.2, 7.9, 10.7, 19.7 and 24.0.

7. The atorvastatin strontium salt or its hydrates or polymorphs according to claim 2, whose X-ray powder diffraction spectrum shows major peaks having I/Iₒ values of at least 10% at 2θ±0.2 of 3.8, 5.2, 5.8, 6.2, 7.6, 8.1, 9.2, 10.3, 11.9, 15.5, 18.1, 19.8, 20.7, 21.1, 22.1, 23.2, 24.3 and 26.3.

8. The atorvastatin strontium salt or its hydrates or polymorphs according to claim 1, which is an amorphous form having no distinctively characteristic peak in the X-ray powder diffraction spectrum thereof.

9. A method for preparing the atorvastatin strontium salt of formula (I) or its hydrates or polymorphs, which comprises (i) bringing atorvastatin of formula (II) or atorvastatin lactone of formula (III) to react with strontium hydroxide; or (ii) adding a reactive strontium salt to atorvastatin sodium or potassium salt to induce salt exchange; or (iii) converting one polymorphic form of the atorvastatin strontium salt or a hydrate thereof previously obtained to other desired polymorphic form:

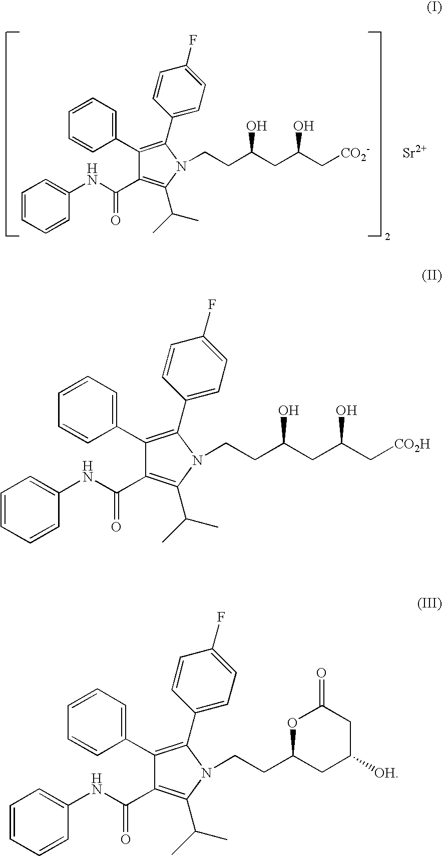

10. The method according to claim 9, wherein the reactive strontium salt is selected from the group consisting of strontium chloride, strontium bromide, strontium sulfate, strontium nitrate, strontium perchlorate, strontium acetate, strontium carbonate, strontium oxalate and a mixture thereof.

11. A pharmaceutical composition for the prevention or treatment of hyperlipidemia and hypercholesterolemia, which comprises the atorvastatin strontium salt or its hydrate or polymorph according to claim 1 as an active ingredient and a pharmaceutically acceptable carrier.

12. The composition according to claim 11, which is a composition for an oral administration.

13. The composition according to claim 12, wherein the amount of the atorvastatin strontium salt, hydrate or polymorph thereof is the range of 0.1 to 95% by weight based on the total composition.

14. The composition according to claim 13, wherein the amount of the atorvastatin strontium salt, hydrate or polymorph thereof is the range of 1 to 70% by weight based on the total composition.
